(19) United States
(12) Patent Application Publication
(10) Pub. No.: US 2015/0190130 A1
(43) Pub. Date: Jul. 9, 2015

(54) DEVICE AND METHOD FOR ANCHORING SOFT TISSUE TO BONE

(71) Applicant: Groh Enterprises, Fairview, NC (US)

(72) Inventor: Gordon I. Groh, Fairview, NC (US)

(21) Appl. No.: 14/590,190

(22) Filed: Jan. 6, 2015

Related U.S. Application Data
(60) Provisional application No. 61/923,853, filed on Jan. 6, 2014.

(51) Int. Cl. A61B 17/04; A61F 2/08

(52) U.S. Cl. CPC A61B 17/0401; A61F 2/0811; A61B 2017/0464; A61B 2017/044; A61B 2017/0445; A61F 2002/0829; A61F 2002/0817

(57) ABSTRACT

A surgical anchor configured to engage soft tissue within a bone cavity defined by a bottom surface and a side surface includes a main body having an exterior surface, a proximal end, and opposing distal end; a tissue capture portion disposed proximate the distal end of the main body; a handle engagement portion disposed proximate the proximate end of the main body; and at least one anchoring member protruding from the exterior surface of the main body. The handle engagement portion is configured to operably engage an insertion handle. The tissue capture portion includes at least one tissue engaging member adapted to secure soft tissue between the anchor and the bottom surface of the bone cavity. At least a portion of the exterior surface of the main body is adapted to secure soft tissue between the anchor and the side surface of the bone cavity.

### CROSS-REFERENCE TO RELATED APPLICATION

This application claims the benefit of U.S. Provisional Application No. 61/923,853, filed on Jan. 6, 2014, which is incorporated by reference in its entirety herein.

### BACKGROUND

1. Field of the Disclosure

The present disclosure relates to medical devices and procedures. More particularly, the present invention relates to devices and methods for attaching, securing, and/or otherwise fixedly connecting soft tissue to a rigid material such as bone.

2. Description of Related Art

Several known surgical devices are known for securing soft tissue (e.g., tendons, ligaments, and/or other soft connective tissue) to bone for various medical procedures, such as orthopedic surgery. Some of these surgical devices function alone to secure the soft tissue to bone, while others may utilize sutures in conjunction with the surgical device to secure the soft tissue to bone, such as a suture anchor.

Suture anchors are designed to secure a soft tissue to a surface of the bone and may not function well for securing soft tissue to a hole that has been drilled within the bone in medical procedures such as anterior cruciate ligament reconstruction, posterior cruciate ligament reconstruction, distal bicep tendon repair, and/or the like.

Other surgical devices that exist for securing soft tissue to bone suffer from other inherent weaknesses, such as being easily removable from a hole that has been drilled within the bone, binding, twisting, and/or wrapping of the soft tissue as the surgical device is placed within the hole drilled within the bone, and/or failing to position the soft tissue into maximum contact with the hole drilled within the bone. Additional difficulties while using those surgical devices may include the inability to fully deploy the soft tissue to the appropriate depth within the hole drilled within the bone while securely fixing the medical device to the bone.

### BRIEF SUMMARY OF THE DISCLOSURE

The present invention consists of several features and a combination of parts hereinafter fully described and illustrated in the accompanying drawings, it being understood that various changes in the details may be made without departing from the scope of the invention or sacrificing any of the advantages of the present invention. The above and other needs are met by aspects of the present disclosure which, in one aspect, provides a surgical anchor configured to secure, engage, and/or otherwise fixedly position soft tissue within a bone cavity that includes a bottom surface and a side surface.

According to one embodiment of the present disclosure the surgical anchor may include a main body having an exterior surface, a proximal end, and a longitudinally opposing distal end. The surgical anchor may further include a tissue capture portion disposed proximate the distal end of the main body, a handle engagement portion defined by the main body and disposed proximate the proximate end of the main body, and at least one anchoring member and/or feature protruding from the exterior surface of the main body. The handle engagement portion may be configured to operably engage an insertion handle. In some embodiments, the tissue capture portion may further include at least one tissue engaging member and/or feature that is adapted to secure at least a first portion of soft tissue between the surgical anchor and the bottom surface of the bone cavity. Further, at least a portion of the exterior surface of the main body of the surgical anchor may be adapted to secure at least a second portion of soft tissue between the surgical anchor and the side surface of the bone cavity.

In some embodiments, the at least one tissue engaging member includes a plurality of protrusions extending from the exterior surface of the main body and/or longitudinally from the distal end of the main body. In another embodiment, the at least one tissue engaging member includes an eyelet adapted to receive a portion of soft tissue and/or a suture therethrough.

According to some embodiments, the tissue capture portion may further define at least a pair of suture channels that extend longitudinally from the distal end of the main body to the handle engagement portion. Further, at least one tissue engaging member may include a suture that extends through a first suture cavity and back through the second suture cavity so as to form a loop disposed at the distal end of the main body.

In some embodiments, the at least one anchoring member includes a threaded surface that extends and/or protrudes about the exterior surface of the anchor. In another embodiment, the at least one anchoring member includes at least one serrated ridge that extends along a circumference of the main body.

According to some embodiments, the main body further includes a handle engaging member that is disposed proximate the handle engagement portion. The handle engaging member may be configured to operably engage the insertion handle. In some embodiments, the handle engaging member may include a threaded surface.

According to another embodiment, the surgical anchor may further include an expansion cavity that extends longitudinally from the handle engagement portion toward the tissue capture portion. The surgical anchor may also include a handle having a proximal end and a distal end, wherein the distal end of the handle defines a handle engagement portion engaging member that is configured to operably engage the handle engagement portion of the main body. The handle may further define an insertion channel that is in communication with the expansion cavity. In some embodiments, the anchor may also include an insertion plug that is disposed within the insertion channel of the handle.

In some embodiments, the anchor may further include a threaded surface that defines the expansion cavity. In another embodiment, the anchor may further include at least one serrated ridge that protrudes from a surface defining the expansion cavity.

In yet another embodiment, the anchor may include an exterior surface of the main body that defines a plurality of spaced slits that extend from the proximal end. The plurality of spaced slits may define a plurality of expansion flaps therebetween, and, in some embodiments, only a portion of the plurality of expansion flaps may include an anchoring member. According to one embodiment, the main body may define at least four equally spaced expansion flaps, wherein the at least one anchoring member protrudes from only two diametrically opposed expansion flaps. These and other features, aspects, and advantages of the disclosure will be apparent from a reading of the following detailed description together with the accompanying drawings, which are briefly described below.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain the principles of the invention. No attempt is made to show structural details of the invention in more detail than may be necessary for a fundamental understanding of the invention and the various ways in which it may be practiced. In the drawings:

FIG. 1 illustrates a perspective view of a surgical anchor according to one embodiment of the present disclosure;

FIG. 2 illustrates a side view of the surgical anchor of FIG. 1 according to one embodiment of the present disclosure;

FIG. 3 illustrates a cross-sectional view of the surgical anchor of FIG. 2 according to one embodiment of the present disclosure;

FIG. 4 illustrates a perspective view of a surgical anchor according to one embodiment of the present disclosure;

FIG. 5 illustrates a perspective view of a surgical anchor according to one embodiment of the present disclosure;

FIG. 6 illustrates a perspective view of a surgical anchor according to one embodiment of the present disclosure;

FIG. 7 illustrates a perspective view of a surgical anchor according to one embodiment of the present disclosure;

FIG. 8 illustrates a cross-sectional view of a surgical anchor according to one embodiment of the present disclosure;

FIG. 9A illustrates a perspective view of a surgical anchor according to one embodiment of the present disclosure;

FIG. 9B illustrates a side view of the surgical anchor of FIG. 9A according to one embodiment of the present disclosure;

FIG. 9C illustrates a perspective view of a surgical anchor according to one embodiment of the present disclosure;

FIG. 9D illustrates a side view of the surgical anchor of FIG. 9C according to one embodiment of the present disclosure;

FIG. 10 illustrates a perspective view surgical anchor according to one embodiment of the present disclosure;

FIG. 11 illustrates a side view of the surgical anchor of FIG. 10 according to one embodiment of the present disclosure;

FIG. 12A illustrates a cross-sectional view of a surgical anchor according to one embodiment of the present disclosure;

FIG. 12B illustrates a cross-sectional view of a surgical anchor according to one embodiment of the present disclosure;

FIG. 13 illustrates a perspective view of a surgical anchor according to one embodiment of the present disclosure; and

FIGS. 14A-14D illustrate a use of a surgical anchor according to one embodiment of the present disclosure.

### DETAILED DESCRIPTION OF THE DISCLOSURE

The aspects of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals represent similar parts throughout the several views of the drawings.

It is understood that the invention is not limited to the particular methodology, devices, apparatus, materials, applications, etc., described herein, as these may vary. It is also to be understood that the terminology used herein is used for the purpose of describing particular embodiments only, and is not intended to limit the scope of the invention. It must be noted that as used herein and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art to which this invention belongs.

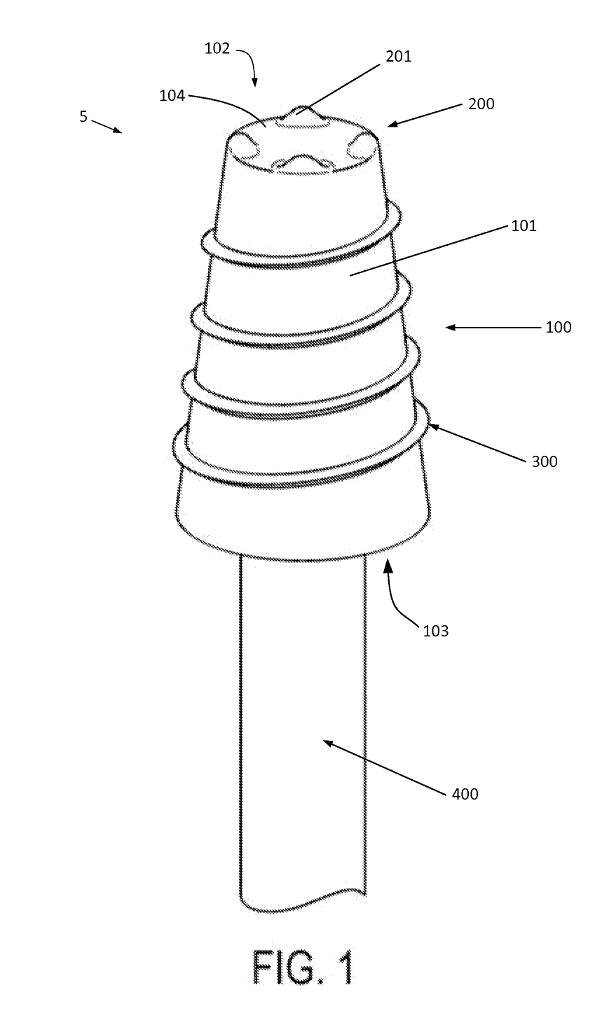

FIGS. 1-14D generally illustrate various embodiments of the invention directed to a surgical anchor. In one embodiment, as shown in FIG. 1, the surgical anchor 5 may include a main body 100 that has an exterior surface 101. The main body 100 of the anchor may extend from a distal end 102 to a longitudinally opposing proximal end 103. In addition, the surgical anchor 5 may include a tissue capture portion 200 disposed proximate the distal end 102 of the main body. According to some embodiments, the surgical anchor 5 may include at least one anchoring member 300 that may protrude from the exterior surface 101 of the main body.

The exact shape and size of the surgical anchor 5 may vary based upon the patient's size, gender, the intended medical procedure, the intended location for placing the surgical anchor and/or the like. According to some embodiments, the main body 100 of the surgical anchor 5, the tissue capture portion 200, and the at least one anchoring member 300 may be sized accordingly for use in orthopedic surgery, and more particularly, in surgeries involving securing soft tissue, such as ligaments, tendons, fascia, skin, fibrous tissues, muscles, and/or the like, to bone. In one embodiment, the surgical anchor may be approximately between 2 mm to 40 mm in length. In another embodiment, the surgical anchor may be approximately between 3 mm to 30 mm in length. A surgical anchor 5, according to embodiments of the present disclosure, can be used in surgeries such as anterior cruciate ligament (ACL) repairs, posterior cruciate ligament (PCL) repairs, distal bicep tendon repairs, and/or the like. Additionally, the surgical anchor may be used in a primary application or in a revision application when a secondary medical operation may be required.

In some embodiments, the main body 100 of the surgical anchor 5, the tissue capture portion 200, and the at least one anchoring member and/or feature 300 may be constructed from a wide array of suitable materials that provide the desired strength and support to securing the soft tissue to bone. Accordingly, the material of the surgical anchor may be a biocompatible and/or physiological inert material. In some embodiments, the surgical anchor may be constructed from a metallic or metal alloy material, such as titanium, stainless steel, related alloys, and/or the like. In another embodiment, the surgical anchor may be constructed from polymers, such as a polymer synthesized from an α-hydroxy acid that is derived from natural amino acids. Other polymers may include poly ether ketone (PEK), polyether ether ketone (PEEK), polyetherimide (ULTEM), ultrahigh molecular weight polyethylene (UHMWPE), polyphenylene, and/or other polymer materials known to those of ordinary skill in the art. A surgical anchor constructed from non-metallic materials may provide some benefits, such as eliminating artifacts from medical imaging scans, such as radiography (X-ray), computed tomography (CT), magnetic resonance imaging (MRI), and/or the like. In another embodiment, the surgical anchor may comprise biodegradable material.

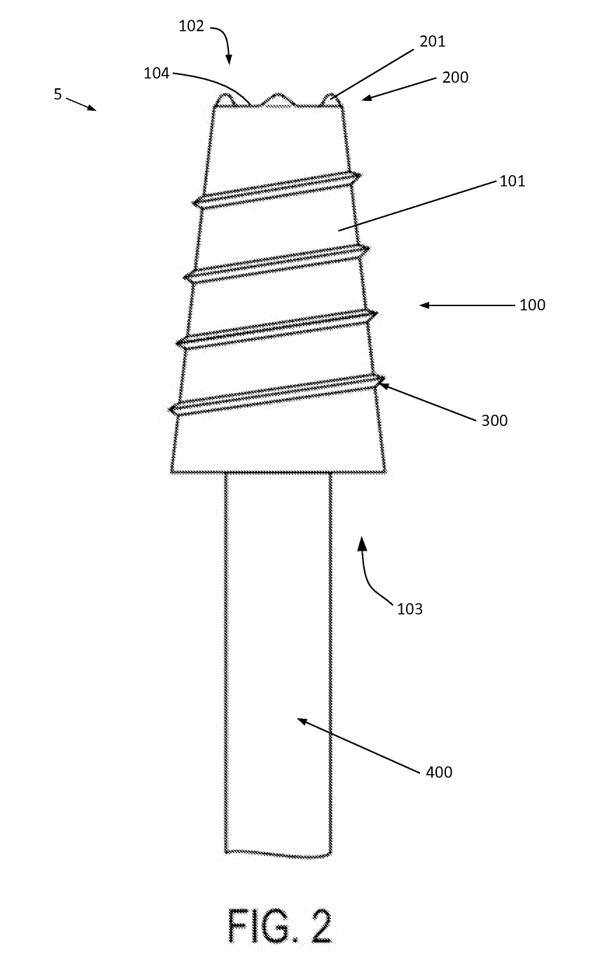

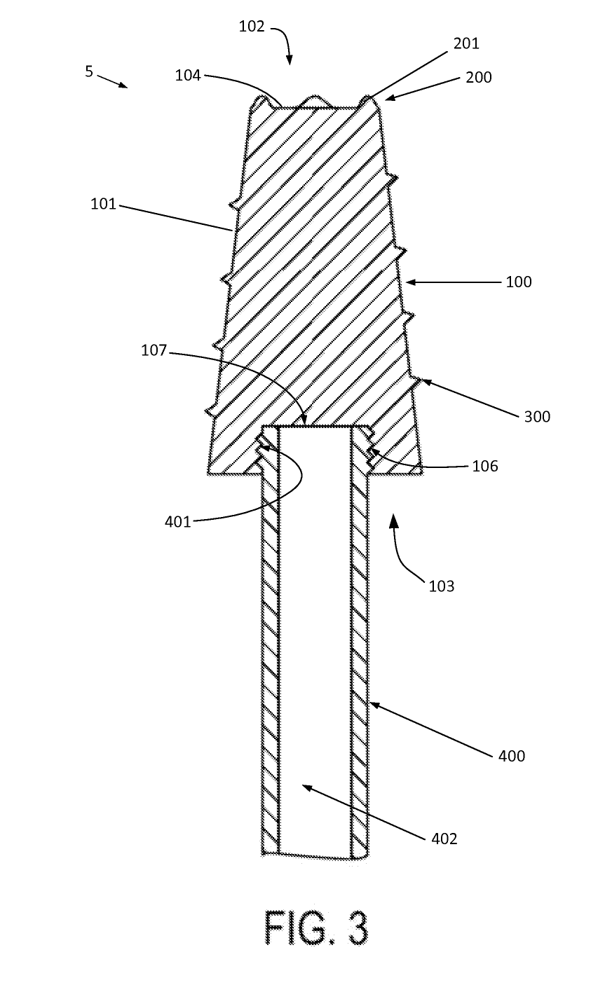

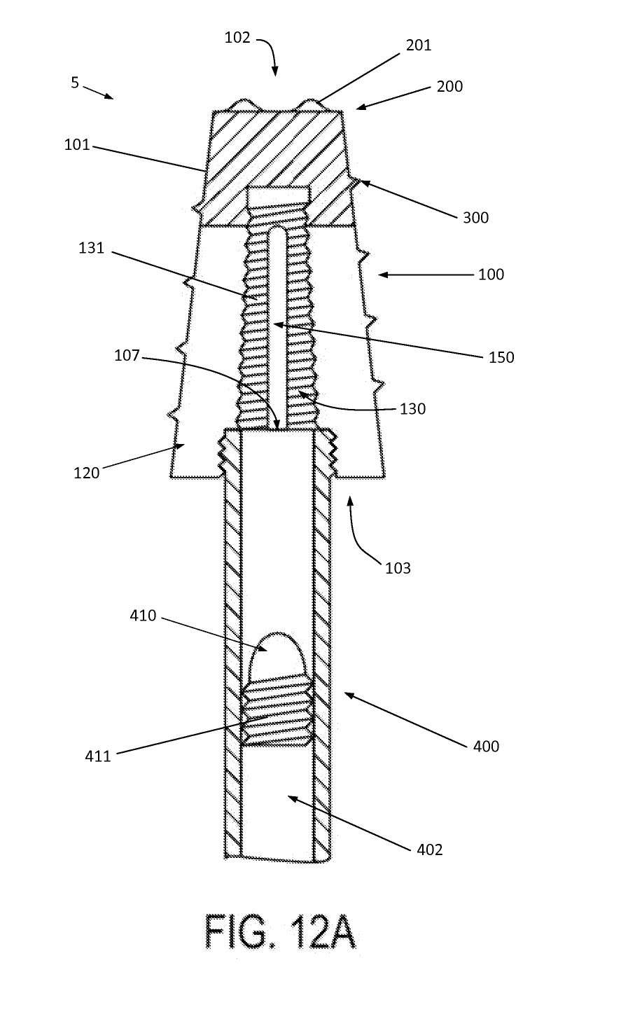

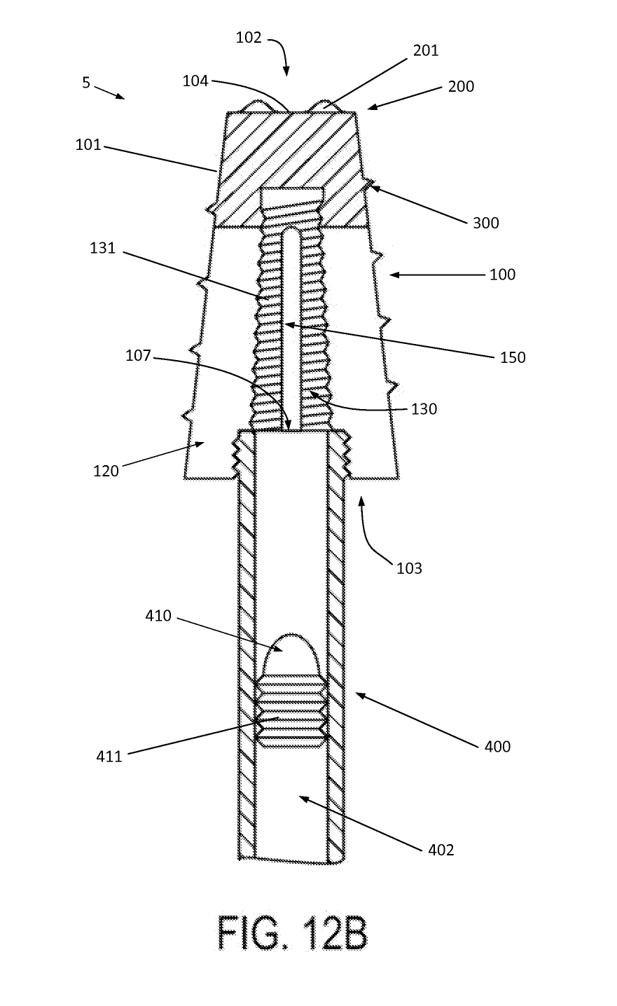

Returning to FIG. 1, a surgical anchor 5 having a main body 100 may be operably engaged with a handle 400. According to some embodiments, the handle 400 may be configured to insert the main body 100 of the surgical anchor within a patient during a medical procedure so as to secure, engage, or otherwise position a portion of a soft tissue within a bone cavity. As shown in FIGS. 1 and 2, the proximal end 103 of the main body 100 may be operably engaged with the handle 400. According to some embodiments, the handle 400 may be a hollow cylinder, which may provide for insertion of a viewing instrument, other surgical instrument, suture, and/or the like therethrough. As shown in the embodiments of FIGS. 3 and 12A, the handle 400 may further define an insertion channel 402 that extends longitudinally from a first end of the handle to an opposing end. In some embodiments, the insertion channel 402 may be configured to provide for the insertion and/or retention of a viewing instrument, other surgical instrument, suture, and/or an insertion plug 410 therein (as shown in FIGS. 12A and 12B).

As illustrated in FIG. 3, the proximal end 103 of the main body 100 may define a handle engagement portion 107 (e.g., a handle cavity) that is configured to receive the handle 400 therein. The handle engagement portion 107 may further define a handle engaging member and/or feature 106 that is configured to operably engage the handle 400 for coupling the handle with the main body of the surgical anchor. In one embodiment, the handle engaging feature 106 may include a threaded surface configured to receive and engage a corresponding threaded surface 401 defined by the handle 400. In another embodiment, the handle engaging feature may include a snap-fit, press-fit, and/or the like engaging feature for coupling the handle with the main body. In yet another embodiment, the handle engaging feature may include magnets configured to couple the handle with the main body. Although illustrated as a threaded connection within a handle cavity in FIG. 3, other engagement features are also contemplated by embodiments of the present disclosure as will be understood by those skilled in the art.

The surgical anchor 5 may further include a tissue capture portion 200 that is disposed proximate the distal end 102 of the main body 100 of the anchor. According to embodiments of the present disclosure, a tissue capture portion 200 may be adapted to secure a portion of soft tissue to the bottom surface of a bone cavity. The tissue capture portion 200 may be configured such that when the tissue capture portion interacts with a portion of soft tissue, the tissue capture portion engages, holds, and/or secures the soft tissue to the main body of the surgical anchor. As such, when the tissue capture portion 200 engages, holds, and/or otherwise secures the soft tissue to the main body of the surgical anchor, the surgical anchor may provide assistance in directing and/or positioning the soft tissue into and/or within a bone cavity.

As shown in FIGS. 1-3, a tissue capture portion 200, according to one embodiment, may be disposed proximate the distal end 102 of the main body 100 of the surgical anchor 5, and may include at least one tissue engaging member 201. In one embodiment, the at least one tissue engaging member and/or feature 201 may include a plurality of protrusions extending longitudinally from the distal end 102 of the main body, as illustrated in FIGS. 1-3. According to another embodiment, the at least one tissue engaging member may include a single protrusion extending longitudinally from the distal end 102 of the main body. Additionally and/or alternatively, the tissue engaging member may be of any size or shape suitable for securing, holding, and/or otherwise engaging a portion of soft tissue, and may be disposed at any location on the distal surface 104. In some embodiments, the tissue engaging member may additionally and/or alternatively include at least one protrusion that extends radially outward from a portion of the exterior surface 101 that is located proximate the distal end 102 of the main body.

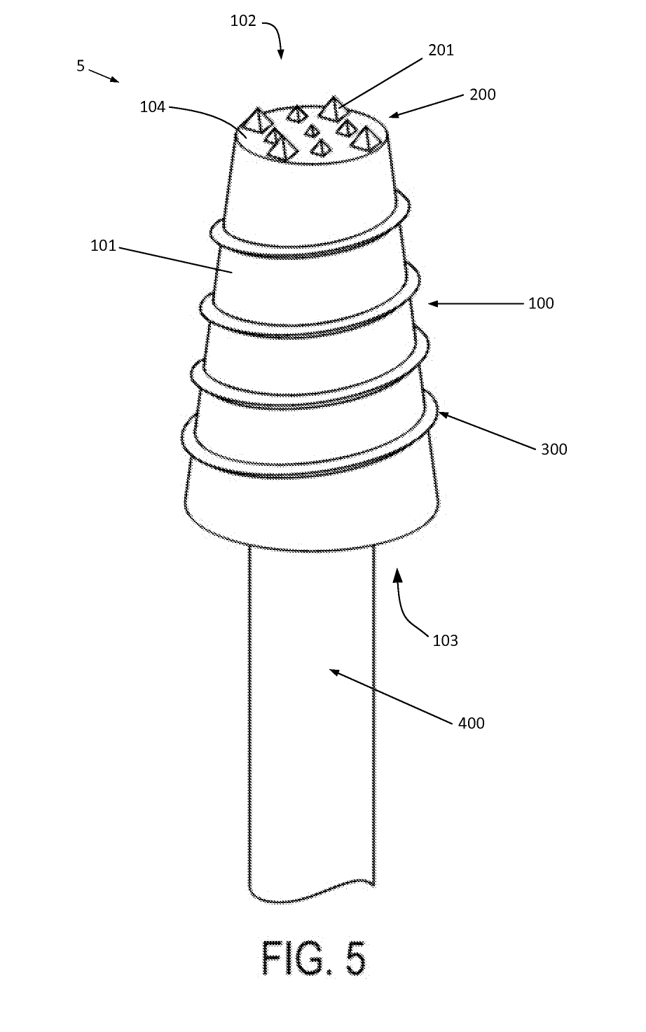

As illustrated in FIG. 1, the tissue engaging member 201 may include a plurality of protrusions that are equally spaced along a circumference proximate the distal end 102 of the main body. Although the illustrated embodiment details four protrusions that are equally spaced along the circumference proximate the distal end of the main body, any number of protrusions are also contemplated by embodiments of the present invention. In addition, the spacing between the protrusions of the tissue engaging feature may vary according to the embodiments. For example, the spacing between the protrusions may not be equally spaced in one embodiment. In another embodiment, the spacing between some of the protrusions may not be equally spaced, while the spacing between another set of protrusions may be equally spaced. According to various embodiments of the present disclosure, the shape of the plurality of protrusions may vary from embodiment to embodiment. As shown in FIG. 5, the plurality of protrusions may be substantially pyramidal in shape. In another embodiment, the plurality of protrusions may be conical in shape. According to yet another embodiment, the plurality of protrusions may be substantially parallelepiped in shape. In one embodiment, the plurality of protrusions may be similarly shaped. Alternatively, in yet another embodiment, each of the plurality of protrusions may be shaped substantially differently from one another. Embodiments of the present invention may contemplate the plurality of protrusions being shaped in any manner that provides for the protrusions to engage at least a portion of the soft tissue so as to anchor and/or position the soft tissue within a bone cavity. Additionally, the plurality of protrusions may be sized in a variety of manners as well. According to one embodiment, the plurality of protrusions may be equally sized with respect to one another, as shown in FIG. 1. In one embodiment, the plurality of protrusions of the tissue engaging feature 201 may all equally extend longitudinally approximately between 1 mm to 10 mm from the distal surface 104 of the main body. In one exemplary embodiment, the plurality of protrusions of the tissue engaging feature 201 may all equally extend longitudinally approximately between 0.1 mm to 5 mm from the distal surface of the main body. In another embodiment, the plurality of protrusions may be sized differently with respect to one another. As shown in FIG. 5, a plurality of protrusions may extend longitudinally in differing lengths with respect to one another. Additionally and/or alternatively, a plurality of protrusions, which are sized differently with respect to one another, may extend outward radially from a portion of the exterior surface 101 that is located proximate the distal end 102 of the main body. Embodiments of the present invention contemplate that the size, shape, configuration, and/or the like of a plurality of protrusions may vary in any manner that provides for the protrusions to engage at least a portion of the soft tissue for anchoring and/or positioning the soft tissue within a bone cavity defined by the bone.

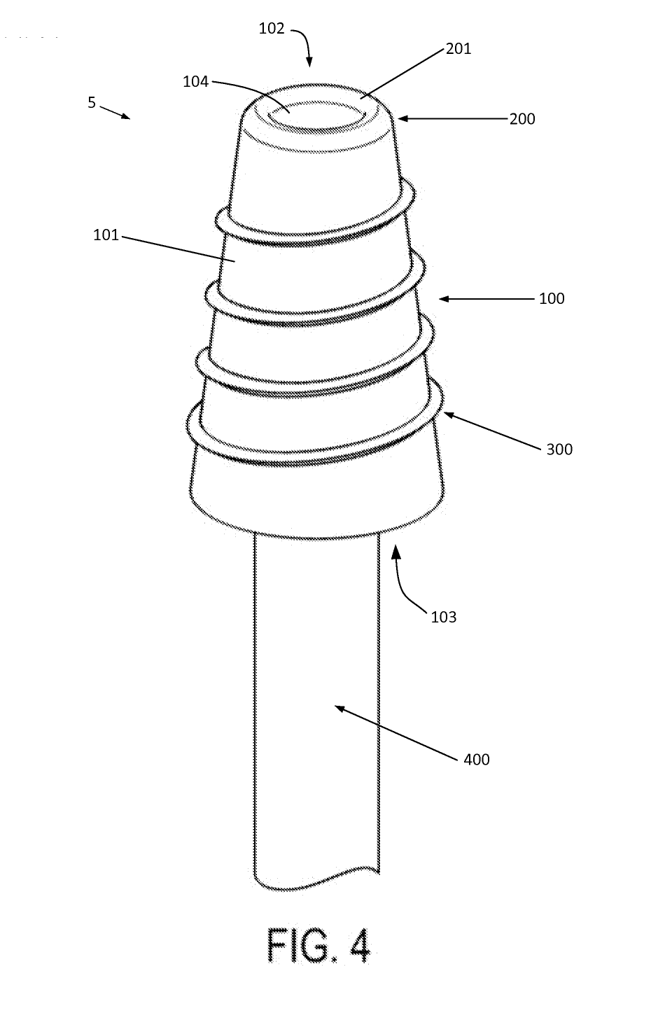

In another embodiment, the tissue engaging feature 201 may include a single protrusion that extends from the distal end 102 of the main body, as shown in FIG. 4. According to one embodiment, the tissue engaging feature 201 may include a protrusion that extends from the distal end of the main body and along a circumference proximate the distal end completely, as shown in FIG. 4. In some embodiments, the tissue engaging feature 201 may be cylindrically shaped with a cross-sectional area that is substantially triangular. Other embodiments of the present invention may contemplate a tissue engaging feature being cylindrically shaped and having a differently shaped cross-sectional area (e.g., square, parabolic, rectangular, etc.).

As illustrated in FIG. 5, the tissue engaging feature 201 may include a number of protrusions that extend longitudinally from the distal end 102 of the main body, and that are shaped and/or sized differently from one another. In one embodiment, the protrusions of the tissue engaging feature 201 may be substantially shaped as differently-sized pyramids extending longitudinally towards a respective apex. The tissue engaging feature 201 may include a first number of protrusions that are sized smaller than other protrusions, and a second number of protrusions that are larger than the first number of protrusions, but are smaller than a third number of protrusions, which may be the largest sized protrusions. Although FIG. 5 illustrates three differently sized protrusions, other embodiments of the present invention may include a tissue engaging feature 201 having any number of differently sized protrusions.

Further, FIG. 5 illustrates the plurality of protrusions of the tissue engaging feature 201 arranged concentrically about an axis that extends from the proximal end to the distal end of the main body. Specifically, the plurality of protrusions may be arranged in concentric squares that are rotated 45 degrees with respect to one another about the axis extending from the proximal end to the distal end. In another embodiment, the plurality of protrusions may be arranged in concentric circles. Although FIG. 5 illustrates the protrusions are arranged in concentric squares that are rotated with respect to one another, embodiments of the present disclosure encompass additional arrangements of the protrusions. Further, FIG. 5 illustrates the smallest protrusions being disposed furthest from the circumference edge, with progressively greater sized protrusions are disposed progressively closer to the circumference edge (i.e., the largest-sized protrusions are located at the circumference edge with the protrusions decreasing in size as the protrusions are placed radially-inward from the circumference edge). In another embodiment, the largest-sized protrusions may be placed at the center of the distal surface 104 and the protrusions of the tissue engaging feature 201 may decrease in size as they are disposed radially outward from the center of the distal surface.

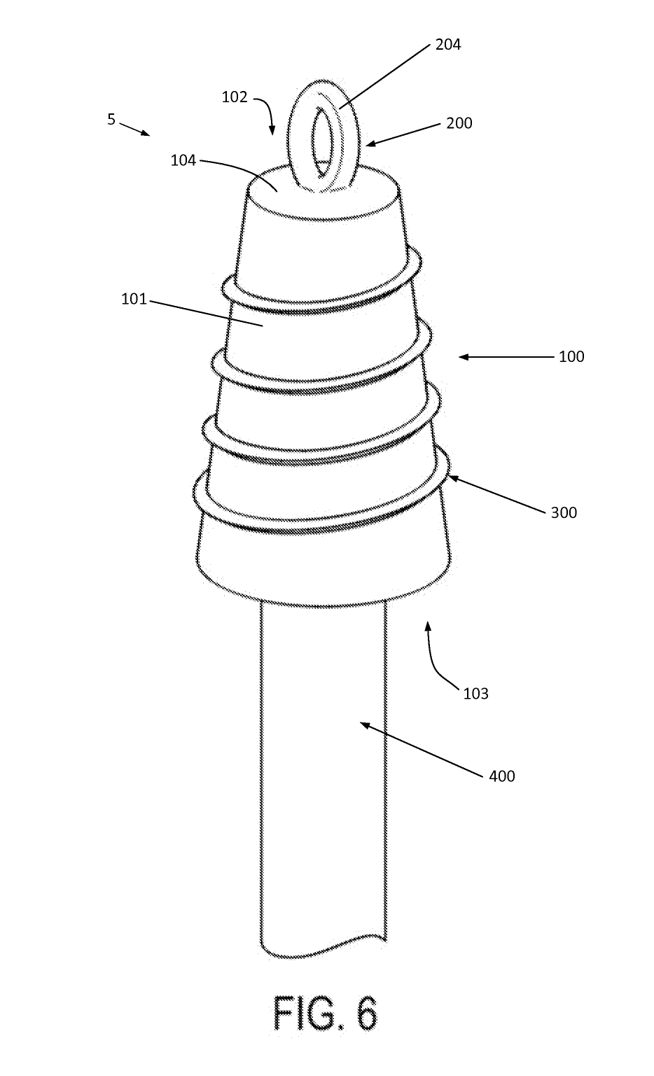

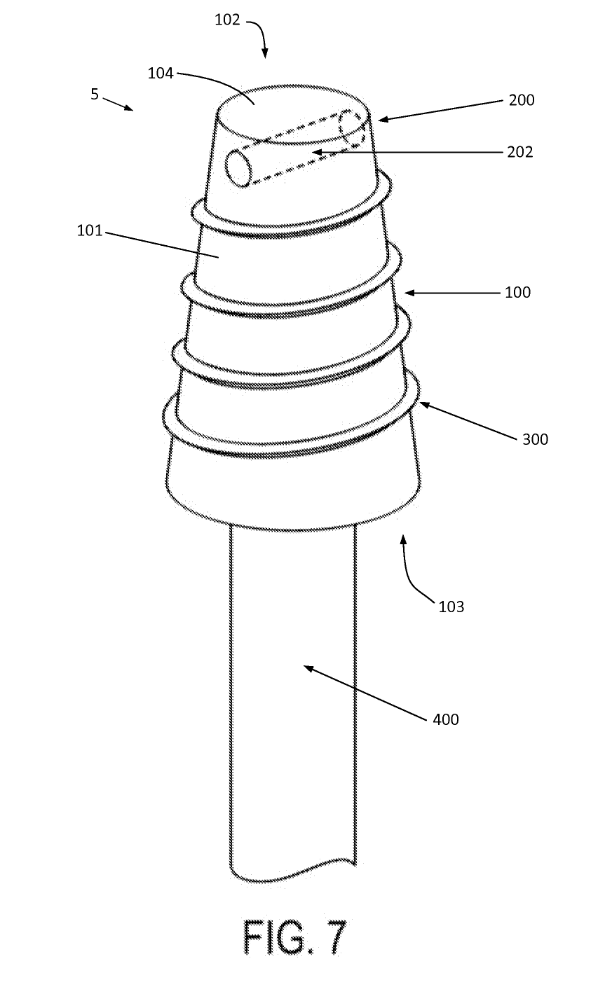

FIG. 6 illustrates another embodiment according to the present disclosure where a tissue engaging feature 201 includes an eyelet 204 configured to receive a portion of a soft tissue and/or a suture therethrough. According to one embodiment, a portion of soft tissue may be folded back upon itself and threaded through the eyelet 204 of the tissue engaging feature 201 so as to provide for a double graft of soft tissue along a single side of the surgical anchor. In another embodiment, the portion of soft tissue may be directly threaded through the eyelet 204 of the tissue engaging feature 201 such that a portion of soft tissue runs along both sides of the main body of the surgical anchor. In yet another embodiment, the eyelet may be configured to receive a suture therethrough for securing a portion of soft tissue to the surgical anchor for placement and/or fixation within the bone cavity. Likewise, FIG. 7 illustrates a channel 202 defined within the tissue capture portion 200 that extends from one side of the exterior surface of the main body to an opposing side. The channel 202 may function in a similar fashion to the eyelet of the tissue engaging feature as shown in FIG. 6. In some embodiments, a surgical anchor may include both the channel 202 and the eyelet of the tissue engaging feature 201.

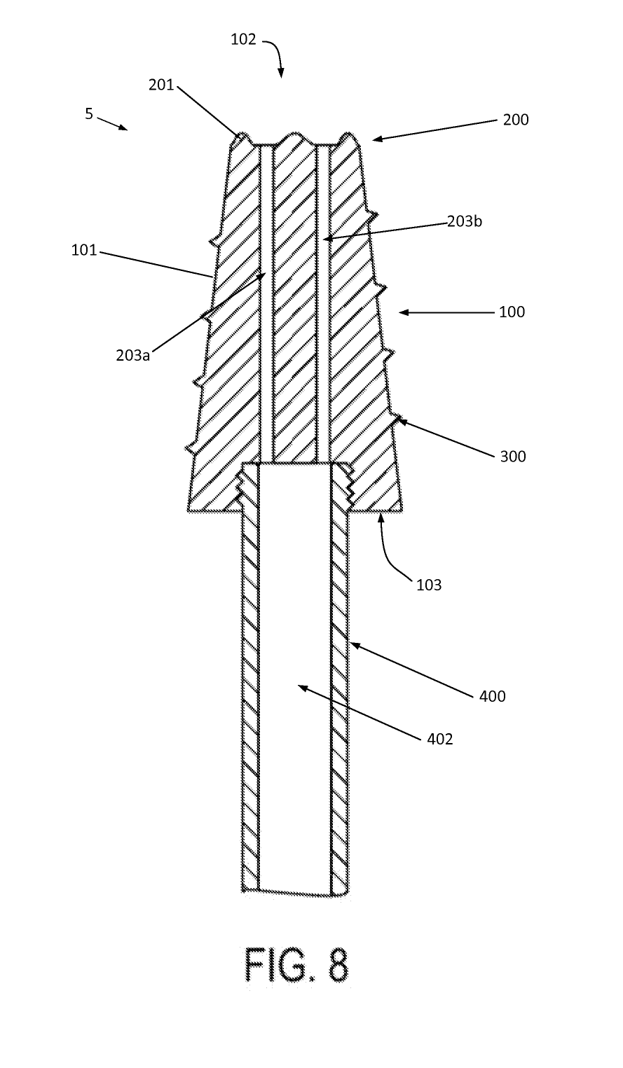

In yet another embodiment, FIG. 8 illustrates a cross-sectional view of a surgical anchor 5 having a main body 100 that defines a plurality of suture channels 203a,203b that extend longitudinally from proximate the distal end of the main body to proximate the proximal end of the main body. The suture channels 203a,203b may be configured to receive a suture therethrough. Specifically, a suture may be threaded from a first suture channel 203a beginning at the proximal end and looped around through the second suture channel 203b beginning at the distal end so as to form a closed loop proximate the distal end of the main body. The suture loop, in conjunction with the tissue capture portion 201, of the surgical anchor may be configured to receive a portion of soft tissue therethrough for placement and/or fixation within a bone cavity.

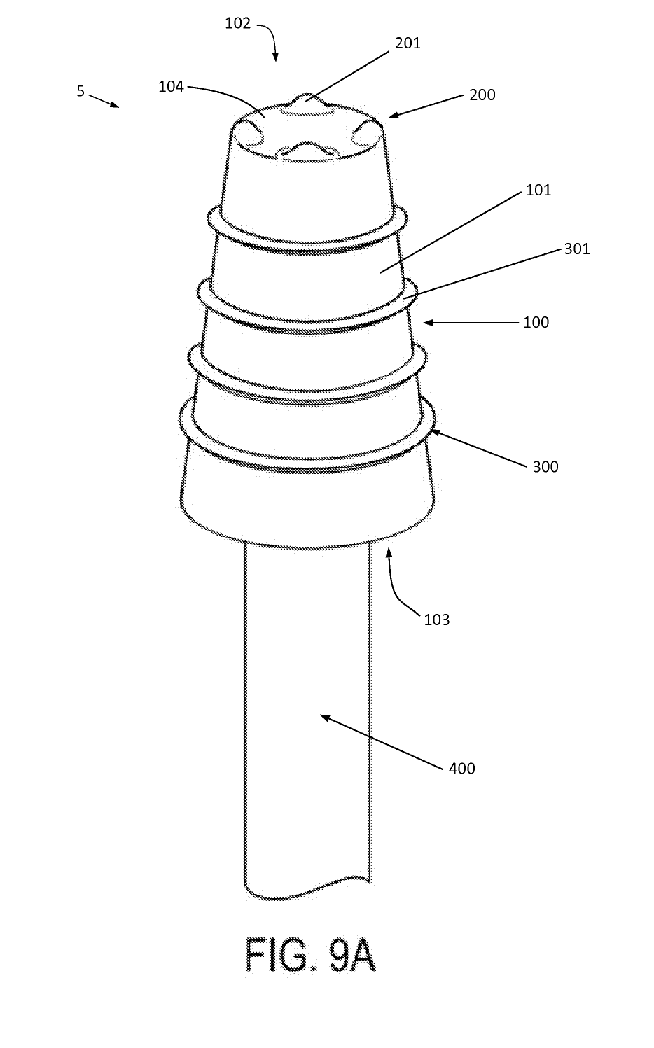

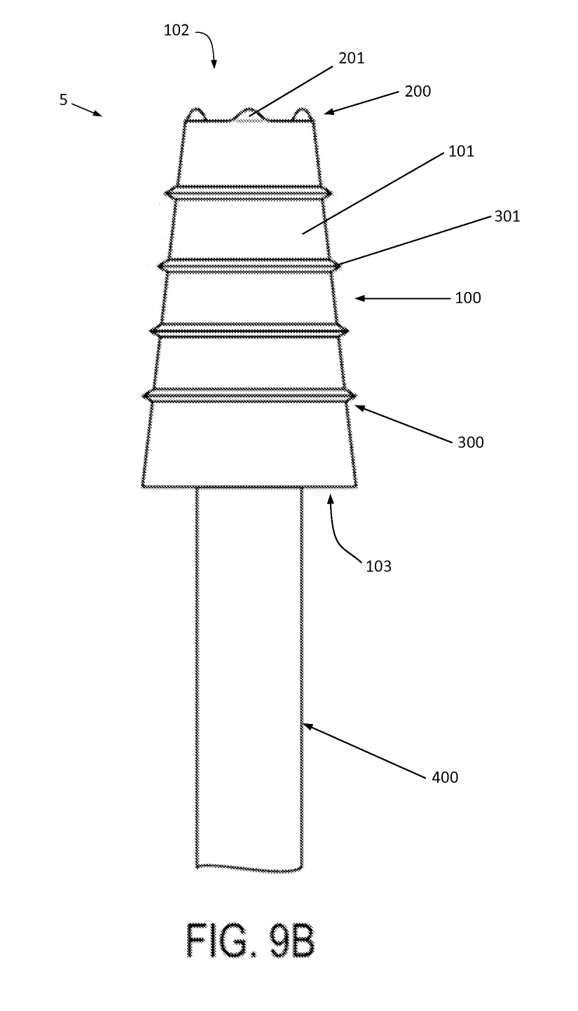

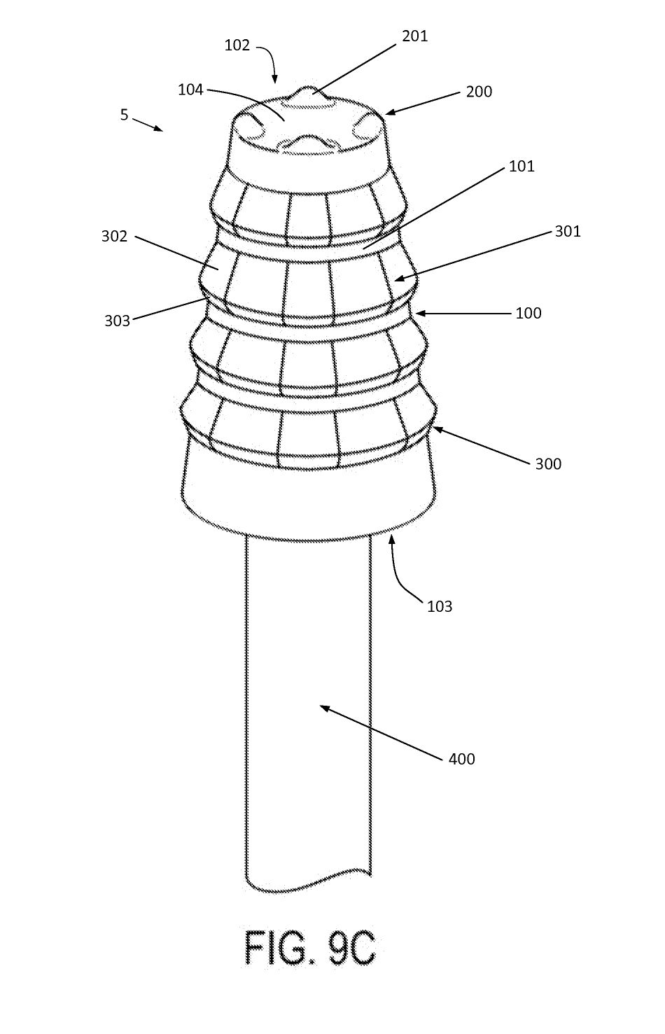

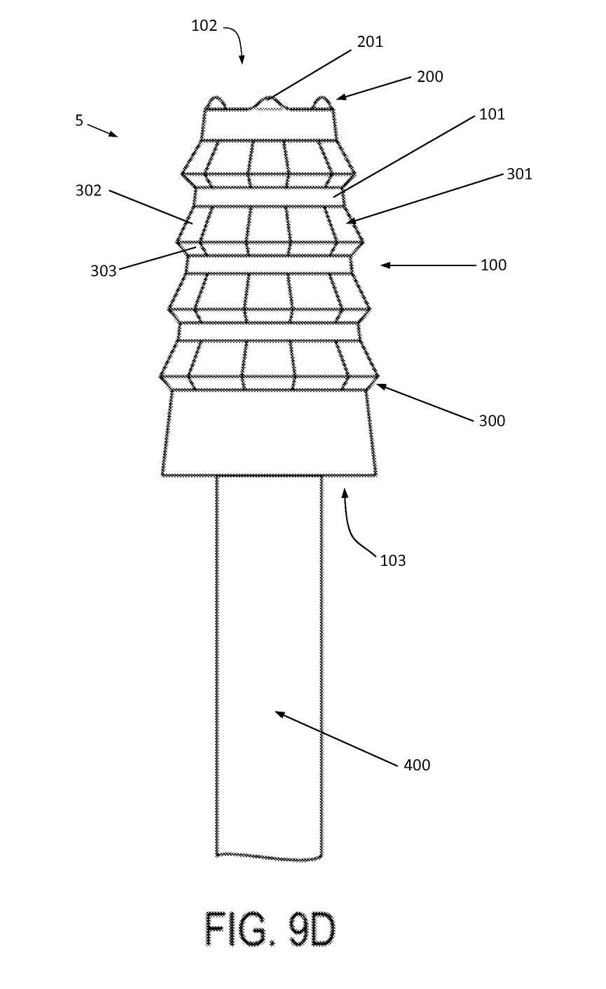

Returning to FIG. 1, the surgical anchor 5 may include at least one anchoring member 300 that protrudes from the exterior surface 101 of the main body 100. According to some embodiments, the anchoring member 300 may include a threaded protrusion configured to fixate the anchor within the bone cavity. In one embodiment, the threaded protrusion may be a unitary piece (e.g., a helical protrusion) that extends about the exterior surface of the main body from the proximal end to the distal end. In another example embodiment, as shown in FIG. 9A-D, the anchoring member 300 may include a plurality of serrated ridges 301 that each extend about the exterior surface of the main body. Like the threaded protrusions, the serrated ridges 301 may be configured to fixate the anchor within the bone cavity when the surgical anchor is placed therein. For example, as illustrated in FIGS. 9C and 9D, the serrated ridge 301 may have a cross-sectional area that is substantially triangular in shape. Further, the serrated ridge 301 may define a first surface 302 that is larger than a second surface 303. The first surface 302 of each serrated ridge 301 may be disposed closer to the distal end of the main body when compared to the respective second surface 303. As such, the first surface 302 of the serrated ridge 301 provides for ease of insertion within the bone cavity, while the second surface 303 provides for greater security and fixation within the bone cavity after insertion of the anchor is complete.

According to another embodiment of the present disclosure, the surgical anchor 5 may further include an expansion cavity 130, as illustrated in FIGS. 12A and 12B. The expansion cavity 130 may extend longitudinally from proximate the proximal end of the main body to proximate the distal end of the main body of the surgical anchor. In another embodiment, the expansion cavity 130 may extend from the handle engagement portion 107 to proximate the distal end 102 of the main body. The expansion cavity 130 may also extend from proximate the handle engagement portion 107 to proximate the tissue capture portion 200 of the surgical anchor.

In some embodiments, the expansion cavity 130 may be substantially cylindrical in shape. According to another embodiment, the expansion cavity 130 may include a tapered cylindrical shape wherein a radius of the expansion cavity measured proximate the distal end of the main body is less than a radius of the expansion cavity measured proximate the proximal end of the main body. The expansion cavity 130 may further be configured to receive an insertion plug 410 therein. For example, the expansion cavity 130 may be shaped such that insertion of the insertion plug 410 therein may cause radial expansion of the surgical anchor 5. In some embodiments, insertion of the insertion plug 410 into the expansion cavity 130 may cause radial expansion of a portion of the main body of the surgical anchor disposed proximate the proximal end of the main body, while a portion of the main body disposed proximate the distal end remains substantially constant in circumference.

FIGS. 12A and 12B further illustrates the expansion cavity 130 defining an interior surface 131 that may include a threaded surface. In one embodiment, the insertion plug 410 may include an engagement member 411 configured to engage the interior surface defining the expansion cavity 130. In some embodiments, the engagement member 411 may include a reciprocally and/or correspondingly threaded surface configured to mate with the threaded surface of the interior surface defining the expansion cavity. In another embodiment, the engagement member and/or feature 411 may include a plurality of serrated ridges configured to engage the interior surface defining the expansion cavity. Although FIG. 12B illustrates one embodiment where the interior surface 131 defining the expansion cavity 130 includes a threaded surface, one of ordinary skill in the art may appreciate that additional and/or alternative embodiments may include an unthreaded surface as the interior surface that defines the expansion cavity. In some embodiments, the interior surface that defines the expansion cavity may be a smooth surface configured to engage with a plurality of serrated ridges of the insertion plug's engagement member. According to another embodiment, the interior surface that defines the expansion cavity may include a plurality of serrated ridges configured to engage the plurality of serrated ridges of the insertion plug's engagement member.

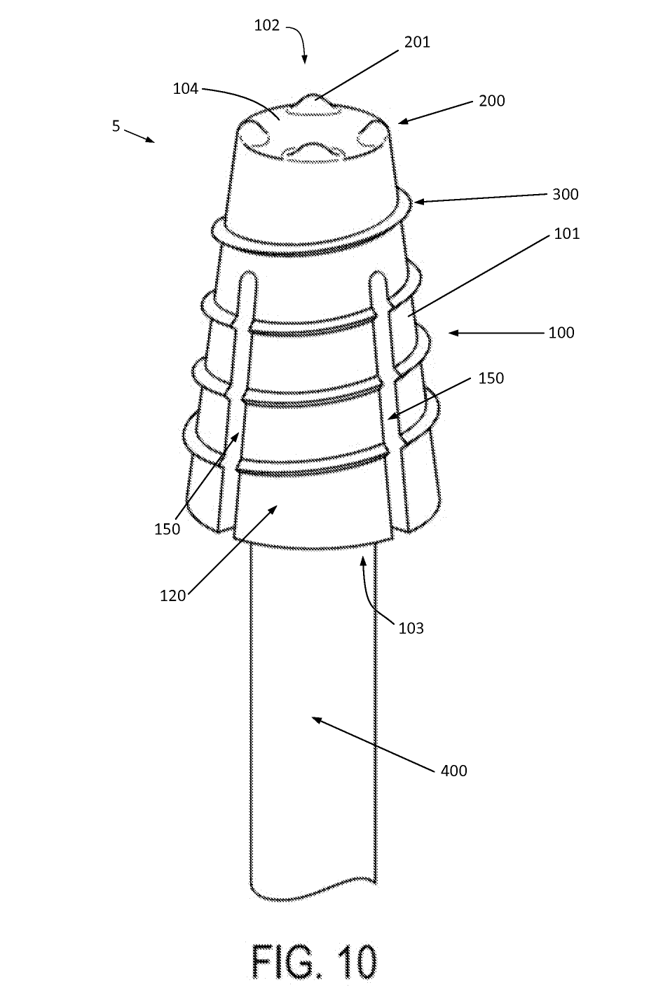

FIG. 10 illustrates a surgical anchor 5 according to one embodiment of the present disclosure that includes a plurality of expansion slits 150 defined by the main body 100 of the surgical anchor. The plurality of expansion slits 150 may extend longitudinally from the proximal end of the main body. In some embodiments, the expansion slits 150 may extend for approximately half the longitudinal distance of the main body. In another embodiment, the expansion slits 150 may extend for approximately less than half the longitudinal distance of the main body. In yet another embodiment, the expansion slits 150 may extend for approximately more than half the longitudinal distance of the main body, as illustrated in FIG. 10.

FIG. 10 illustrates a surgical anchor 5 including a plurality of expansion slits 150, wherein a pair of adjoining expansion slits 150 defines an expansion flap 120 therebetween. For example, FIG. 10 illustrates an embodiment of the present disclosure of a surgical anchor that includes four expansion slits. Since each of the pairs of adjoining expansion slits define a respective expansion flap 120 therebetween, the particular embodiment illustrated in FIG. 10 includes four expansion flaps 120. Although embodiments illustrated herein include a surgical anchor having four expansion slits and a respective four expansion flaps, any number of expansion slits and the corresponding number of expansion flaps are encompassed within this disclosure.

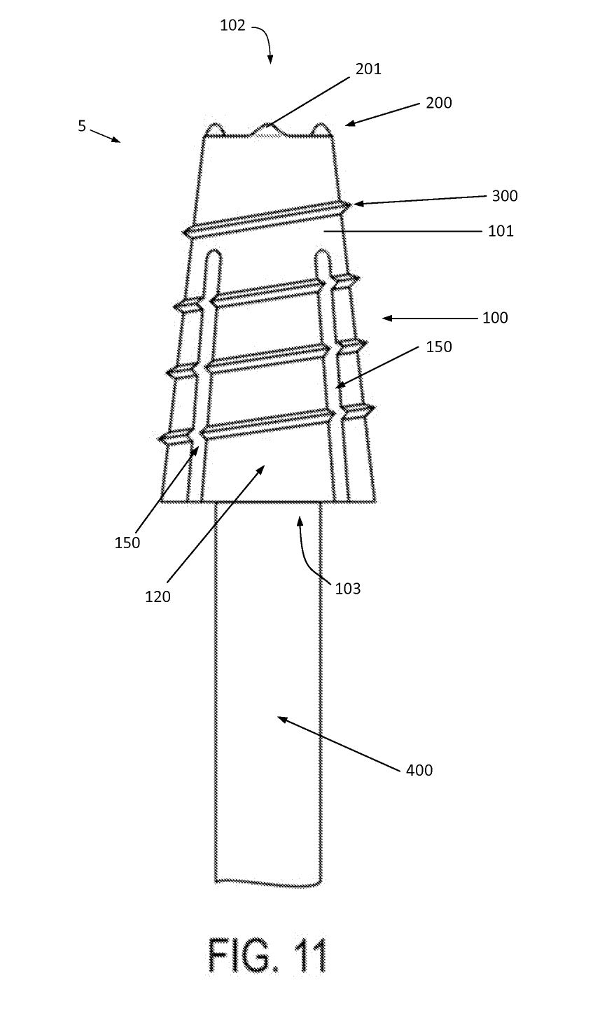

FIGS. 10 and 11 further illustrates an anchoring member and/or feature 300 that includes a threaded portion that extends about the exterior surface 101 of the main body 100 of the surgical anchor. According to some embodiments, the expansion slits 150 may intersect the anchoring feature without limiting the functionality of the anchoring feature. As such, although the openings provided by the expansion slits traverse the anchoring feature 300, the threaded portions of the anchoring feature still provide for securing and fixating the surgical anchor, as well as a portion of the soft tissue, within the bone cavity.

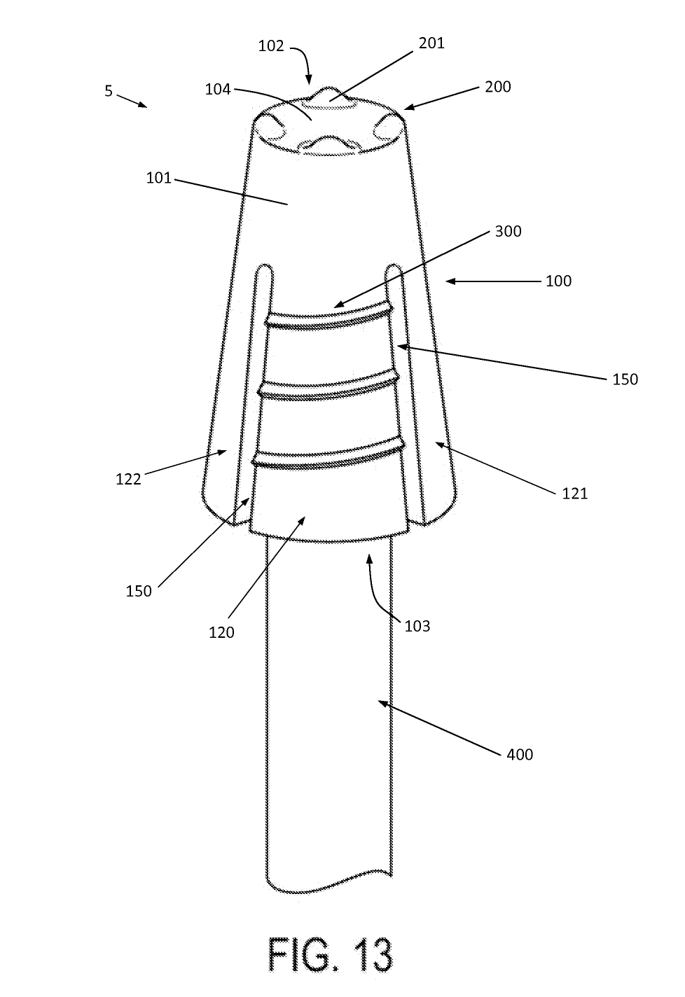

FIG. 13 illustrates another example embodiment of the present invention that includes a plurality of expansion flaps 120. Unlike previous embodiments illustrated in FIGS. 10-12B, an anchoring feature 300 may be disposed only on a subset of a plurality of expansion flaps 120, as shown in FIG. 13. For example, as shown in FIG. 13, a first expansion flap 120 and a second expansion flap (not shown) radially opposed to the first expansion flap may include an anchoring feature 300, while a third and fourth expansion flap 121,122, disposed between the first and second expansion flaps, may lack any anchoring feature 300. As such, the third and fourth expansion flaps 121,122 may provide a substantially smooth exterior surface that provides for relatively smooth insertion of the soft tissue into the bone cavity. Further, the smooth exterior surfaces of the third and fourth expansion flaps 121,122 may also provide for increased contact area between the surgical anchor, the soft tissue, and the surfaces of the bone cavity. Increased contact between the soft tissue and the surface of the bone cavity may provide for increased probabilities in desired outcomes, decreased healing times, and decreased odds for a secondary medical procedure.

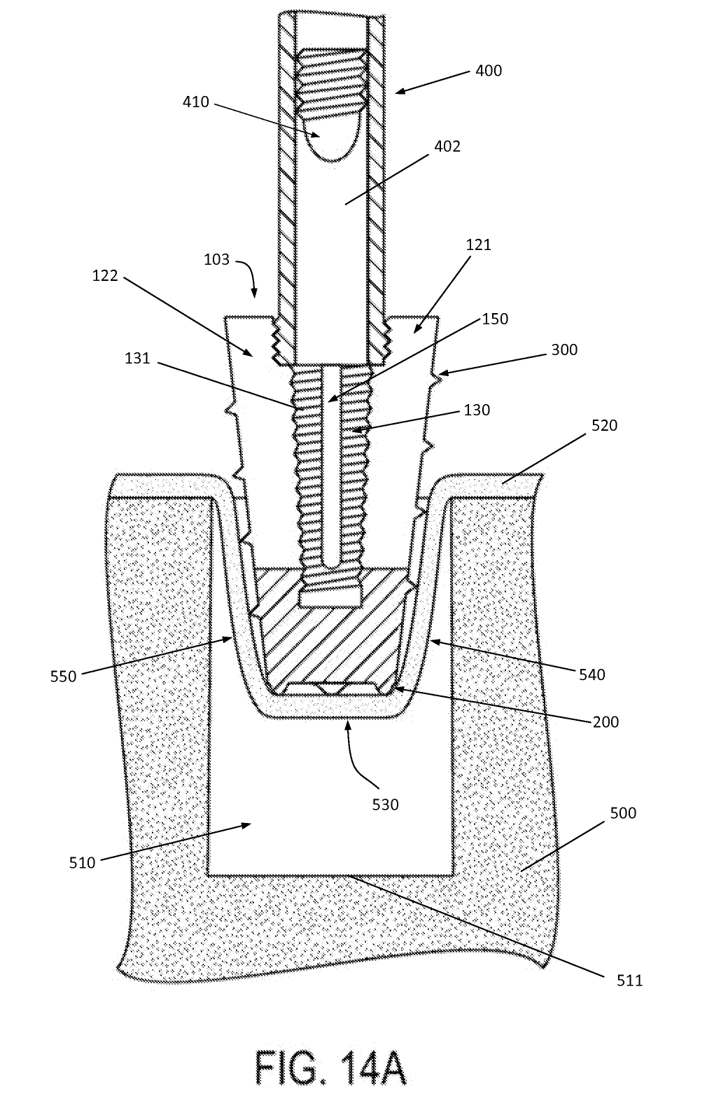

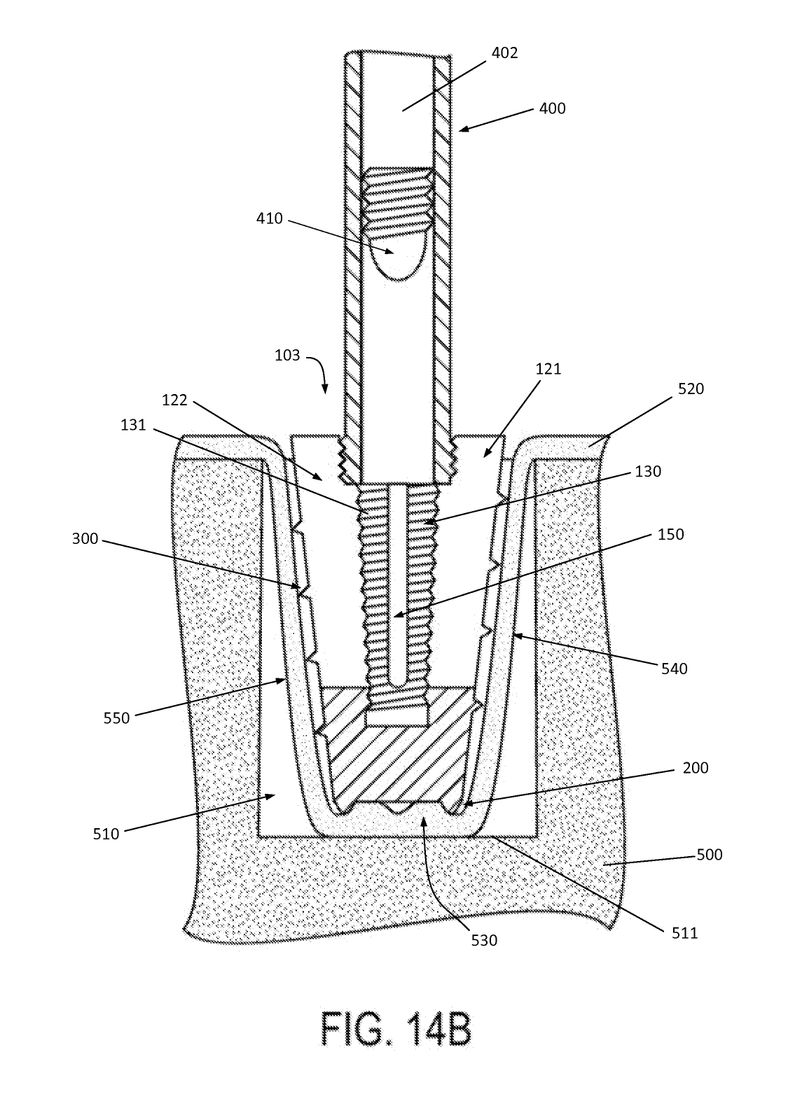

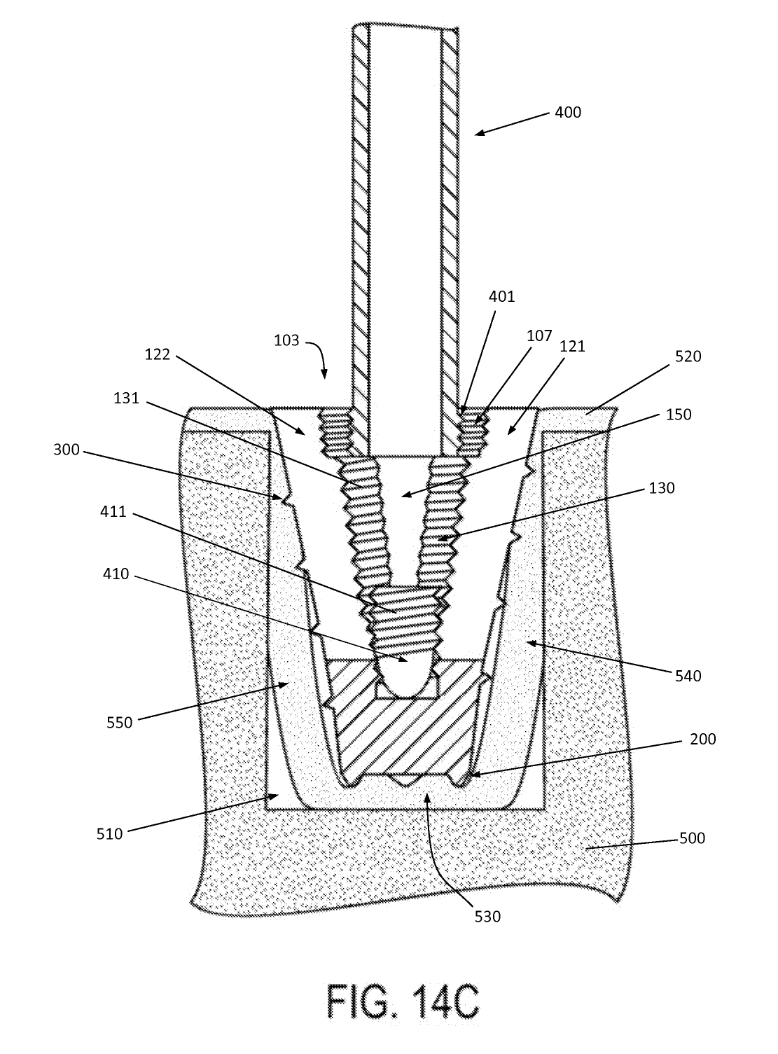

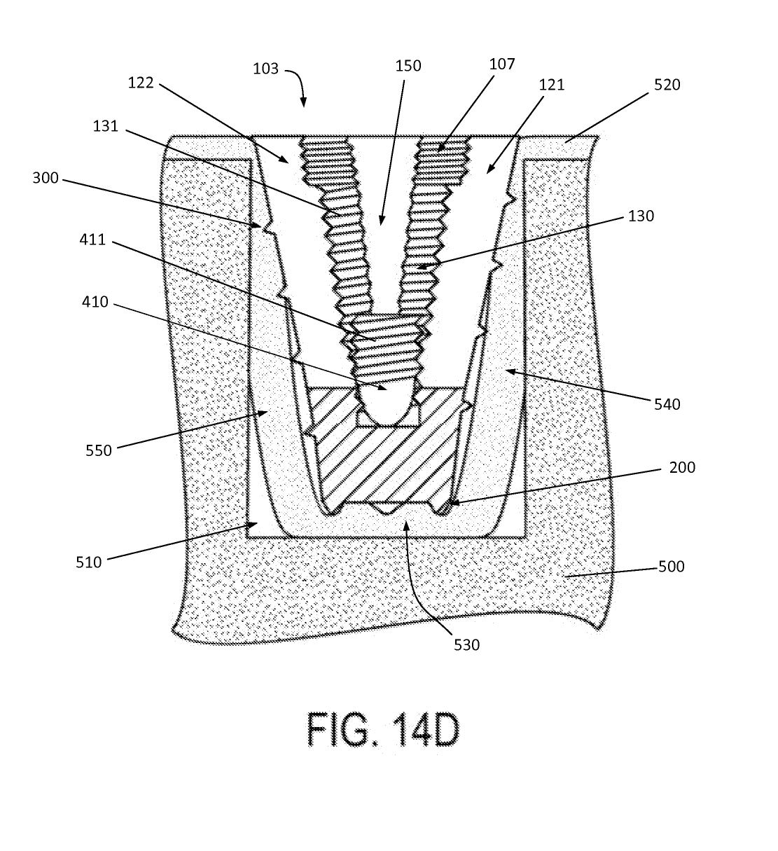

FIGS. 14A-14D illustrate a method of using a surgical anchor 5 according to one embodiment of the present disclosure to affix and/or otherwise securely attach a portion of soft tissue 520 within a bone cavity 510 defined by a bone 500. Further, although FIGS. 14A-14D illustrate a method of using a surgical anchor 5 that includes one particular embodiment of the tissue capture portion 200, methods of using the surgical anchor 5 are not limited to using a tissue capture portion as illustrated in FIGS. 14A-14D, and may include a tissue capture portion according to any of the embodiments discussed herein. As illustrated in FIG. 14A, a surgical anchor 5 may include a tissue capture portion 200 disposed on a distal end of the main body that is engaged with a first portion 530 of soft tissue. The first portion 530 of soft tissue may otherwise be securely engaged with the tissue capture portion 200 such that insertion of the surgical anchor 5 within the bone cavity with the handle 400 urges the first portion of the soft tissue towards a bottom surface 511 of the bone cavity, as shown in FIG. 14A.

In FIG. 14B, the surgical anchor 5 has been fully inserted within the bone cavity 510 such that the first portion 530 of the soft tissue is in maximum contact with the bottom surface 511 of the bone cavity. Although the surgical anchor has been initially affixed within the bone cavity, embodiments of the present disclosure provide for additional securing of the surgical anchor within the bone cavity. For example, insertion of the insertion plug 410 within the hollow cylindrical handle 400 into the expansion cavity 130 may provide for increased security of the surgical anchor within the bone cavity.

According to one embodiment, when the insertion plug 410 is inserted within the expansion cavity 130, the tapered shape of the expansion cavity 130 may bias the plurality of expansion flaps 121, 122 radially outwards, while the tissue capture portion remains relatively unchanged in shape and dimension, as illustrated in FIG. 14C. As such, the plurality of expansion flaps may each urge a respective portion of soft tissue towards a side surface of the bone cavity. Specifically, a first and second expansion flap 121,122 may assist in urging a second portion 540 and a third portion 550 of the soft tissue respectively towards the side surface of the bone cavity. Additionally, a third and fourth expansion flap (not shown) may further assist in urging a second portion 540 and a third portion 550 of the soft tissue towards the side surface of the bone cavity.

In another embodiment, the expansion cavity 130 may be asymmetrically tapered with respect to the expansion flaps such that insertion of the insertion plug 410 therein may cause particular expansion flaps to expand radially outward in a greater amount than other expansion flaps. For example, the expansion cavity 130 in one embodiment may be shaped as a tapered substantially elliptical cylinder such that insertion of an insertion plug 410 therein may cause the expansion flaps disposed proximate the semi-minor axis of the tapered elliptical cylinder to be displaced radially outward in a greater amount than the expansion flaps disposed proximate the semi-major axis of the tapered elliptical cylinder. In another embodiment, the expansion flaps disposed proximate the semi-minor axis of the tapered elliptical cylinder may include anchoring features 300 on the exterior surface thereof, while the expansion flaps disposed proximate the semi-major axis of the tapered elliptical cylinder may have a substantially smooth exterior surface. Accordingly, the anchoring features 300 may be urged further towards the surfaces of the bone cavity to provide for additional security and fixation within the bone cavity. In another embodiment, the expansion flaps disposed proximate the semi-minor axis of the tapered elliptical cylinder may be substantially smooth, while the expansion flaps disposed proximate the semi-major axis of the tapered elliptical cylinder may include anchoring features 300 on the exterior surface thereof. As such, the expansion flaps disposed proximate the semi-minor axis of the tapered elliptical cylinder in this particular embodiment may provide for increased surface contact of the soft tissue with a surface of the bone cavity as those particular expansion flaps are urged outwards to a greater degree than the expansion flaps disposed proximate the semi-major axis. Although illustrated as a substantially tapered cylindrical cavity and a cylindrical bullet respectively, additional shapes and configurations of the expansion cavity and insertion plug are also encompassed within embodiments of the present disclosure.

Further, insertion of the insertion plug 410 within the expansion cavity 130 may also subsequently cause the handle engagement portion 107 to expand radially, as shown in FIGS. 14C and 14D. Accordingly, when the insertion plug has been inserted into the expansion cavity 130, a handle engaging member and/or feature 106 defined by the handle engagement portion 107 may operably disengage from a reciprocal threaded surface 401 defined by the handle 400. As such, insertion of the insertion plug 410 within the expansion cavity 130 provides for an easier and quicker removal of the handle 400 from the handle engagement portion 107. FIG. 14D illustrates a final position of the surgical anchor 5 according to one embodiment where an insertion plug 410 has been inserted into a final depth within the expansion cavity 130 such that a plurality of expansion flaps 150 have expanded radially outwards to provide for greater fixation and attachment of the surgical anchor within a bone cavity.

Many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed herein and that modifications and other embodiments are intended to be included within the scope of the appended claims. Although specific terms are employed herein, they are used in a generic and descriptive sense only and not for purposes of limitation.

That which is claimed:

1. A surgical anchor configured to engage soft tissue within a bone cavity that includes a bottom surface and a side surface, the anchor comprising:
a main body having an exterior surface, a proximal end, and a longitudinally opposing distal end;
a tissue capture portion disposed proximate the distal end of the main body;
a handle engagement portion disposed proximate the proximate end of the main body; and
at least one anchoring member protruding from the exterior surface of the main body,
wherein the handle engagement portion is configured to operably engage an insertion handle,
the tissue capture portion further comprises at least one tissue engaging member adapted to secure at least a first portion of soft tissue between the surgical anchor and the bottom surface of the bone cavity, and
at least a portion of the exterior surface of the main body is adapted to secure at least a second portion of soft tissue between the surgical anchor and the side surface of the bone cavity.

2. The anchor of claim 1, wherein the at least one tissue engaging member comprises a plurality of protrusions extending from the exterior surface of the main body and/or longitudinally from the distal end of the main body.

3. The anchor of claim 1, wherein the at least one tissue engaging member comprises an eyelet adapted to receive a portion of soft tissue and/or a suture therethrough.

4. The anchor of claim 1, wherein the tissue capture portion further defines at least a pair of suture channels extending longitudinally from the distal end of the main body to the handle engagement portion, and
wherein at least one tissue engaging member comprises a suture extending through a first suture cavity and back through the second suture cavity so as to form a loop disposed at the distal end of the main body.

5. The anchor of claim 1, wherein the at least one anchoring member comprises a threaded surface extending about the exterior surface of the anchor.

6. The anchor of claim 1, wherein the at least one anchoring member comprises at least one serrated ridge extending along a circumference of the main body.

7. The anchor of claim 1, wherein the main body further comprises a handle engaging member disposed proximate the handle engagement portion, the handle engaging member configured to operably engage the insertion handle.

8. The anchor of claim 7, wherein the handle engaging member includes a threaded surface.

9. The anchor of claim 1, further comprising:
an expansion cavity, the expansion cavity extending longitudinally from the handle engagement portion toward the tissue capture portion;
a handle having a proximal end and a distal end, wherein the distal end of the handle defines a handle engagement portion engaging member that is configured to operably engage the handle engagement portion of the main body, the handle further defining an insertion channel that is in communication with the expansion cavity; and
an insertion plug disposed within the insertion channel of the handle.

10. The anchor of claim 9, further comprising a threaded surface that defines the expansion cavity.

11. The anchor of claim 9, further comprising at least one serrated ridge protruding from a surface defining the expansion cavity.

12. The anchor of claim 9, wherein the exterior surface of the main body further defines a plurality of spaced slits extending from the proximal end, the plurality of spaced slits defining a plurality of expansion flaps therebetween.

13. The anchor of claim 12, wherein only a portion of the plurality of expansion flaps include an anchoring member.

14. The anchor of claim 12, wherein the main body defines at least four equally spaced expansion flaps, and wherein the at least one anchoring member protrudes from only two diametrically opposed expansion flaps.
